(19) United States
(12) Patent Application Publication
(10) Pub. No.: US 2025/0319004 A1
(43) Pub. Date: Oct. 16, 2025

(54) Treatment and composition for reversing the biological age of skin

(71) Applicant: Arri Russell Morris, Austin, TX (US)

(72) Inventor: Arri Russell Morris, Austin, TX (US)

(21) Appl. No.: 18/635,850

(22) Filed: Apr. 15, 2024

(51) Int. Cl. A61K 8/365; A61K 8/35; A61K 8/67; A61K 8/96; A61Q 19/02; A61Q 19/08

(52) U.S. Cl. CPC A61K 8/365; A61K 8/35; A61K 8/671; A61K 8/675; A61K 8/678; A61K 8/965; A61Q 19/02; A61Q 19/08; A61K 2800/522

(57) ABSTRACT

Novel methods and compositions for treating aged and environmentally damaged skin are disclosed which measurably reverse the biological, epigenetic age of the skin, resulting in improvements in the skin's visual appearance, function, and biophysical properties The disclosed treatment methods involve topical application of a novel cosmetic composition containing the combination of a cell-membrane stabilizing agent, a metabolic enhancing agent, amino acids, minerals, and nutrients. To some extent, the formulation goes further to achieve restorative results for certain types of skin damage beyond the typical concerns of wrinkles, looseness, age spots, and sun damage.

### STATEMENT REGARDING FEDERALLY SPONSORED RESEARCH OR DEVELOPMENT

Not applicable

### RELATED APPLICATIONS

US 2021/0038729 A1 Feb. 2021 Zonari et al.
US 2016/0222448 A1 Aug. 2016 Stefan Horvath

### Other Publications

Chin, Toby, Yaelim Lee, and Oliver Dreesen. “The role of cellular senescence in skin aging and age-related skin pathologies.” Frontiers in physiology 14 (2023): 1297637.
Naeini, Saghi Hakimi, et al. “Alpha-ketoglutarate as a potent regulator for lifespan and healthspan: Evidences and perspectives.” Experimental Gerontology 175 (2023): 112154.
Stasiulewicz, Marcin, et al. “Mechanism of Osmolyte Stabilization-Destabilization of Proteins: Experimental Evidence.” The Journal of Physical Chemistry B 126.16 (2022): 2990-2999.
Kidd, Parris. “Astaxanthin, cell membrane nutrient with diverse clinical benefits and anti-aging potential.” Altern Med Rev 16.4 (2011): 355-364.

### BACKGROUND

Skin is the largest organ of the human body. Skin serves as the primary barrier and interface between the human body and the external environment. It is widely accepted that the Hayflick limit, dehydration, inadequate nutrition, loss of collagen content, and sun exposure, or other forms of ultraviolet light exposure play key roles in the aging process of skin. An optimal anti-aging formulation must address these issues.

A popular approach to the challenge of aging cells is to simply increase skin cell turnover. Creams containing active ingredients such as urea or vitamin C achieve their anti-aging properties, at least in part, by causing dead and damaged skin cells to be replaced by new ones more quickly. This approach can increase production of collagen and, depending on the active ingredient, may also address nutritional deficiencies, and sun damage. However, this approach completely ignores the Hayflick limit; leading to skin irritation, particularly when higher concentrations are used. Removing older cells may also have limited efficacy in younger subjects and those who already use exfoliants.

The ideal approach would aim to salvage senescent and pre-senescent cells by undoing the most common types of damage. Astaxanthin is known to stabilize damaged cell membranes. Glycine and trimethylglycine have been shown to positively influence the stability of proteins that degrades with age. Supporting collagen production with amino acids reverses wrinkles, which recently have been implicated in acceleration of the overall aging process. Alpha-ketoglutarate reignites cellular metabolic processes and further optimizes epigenetic expression. Vitamins and minerals round off this approach, making up for nutritive deficiencies that can advance the aging process.

### BRIEF DESCRIPTION OF DRAWINGS

FIG. 1 shows the effects of various commercially available creams and the present invention, labeled “Muse 28”, on ex-vivo skin samples after five days of treatment, applied twice per day.

FIG. 2 shows in-vivo resolution of damaged skin after two months of treatment. The subject elected to limit the area of treatment, just applying cream to the damaged spot.

FIG. 3 shows the histology results of ex-vivo skin samples after one week of treatment, applied once per day. No DNA methylation data was collected for this initial study.

### DETAILED DESCRIPTION

The object of the present invention is to provide a novel method of treatment and a novel composition for treating aged and environmentally damaged or deteriorated skin. The present invention provides a method of treating skin comprising topical application to damaged skin of a cosmetic composition in an amount effective to reverse the biological, epigenetic age of the skin, wherein said reversal is effective to induce repair, replacement, and remodeling of the stratum corneum, epidermis, and dermis of the skin and improvements in the function, and overall appearance of the skin.

According to the present invention, “reversing the biological, epigenetic age of skin” is a decrease measured by the Horvath epigenetic clock, described in US20160222448A1, which relies on DNA methylation to calculate the age of various biological samples, including skin.

The composition of the present invention can be used in many cosmetic products including, but not limited to, creams and lotions, gels, ointments, foundation, lipstick, cleansers, toners, masks, and color cosmetic products. The composition is preferably used in anti-aging products for the skin, especially leave-on products.

The cosmetic composition of the present invention is effective at pH values between about 3.0 and about 8.0. Preferably, the pH of the composition is about 4, as a moderately acidic environment is most conducive to regenerative processes. One of ordinary skill in the art may add appropriate pH adjusting ingredients to the compositions of the present invention to adjust the pH to an acceptable range.

The cosmetic composition should be topically applied to any area of the skin requiring treatment with the frequency and in the amount necessary to achieve the desired results. Preferably, the cosmetic composition is applied at least once per day, most preferably twice per day. The frequency of treatment depends on the degree of damage or deterioration of the skin, the responsiveness of the user's skin, the strength of the active ingredients in the cosmetic product, the effectiveness of the vehicle used to deliver the active ingredients, the ease with which the formula is removed by physical contact with clothing or its removal by sweat or other intrinsic or extrinsic fluids, and the convenience to the user's lifestyle. Typical concentrations of the novel treatment composition described herein range from about 0.01% to about 5.0% by weight based on the total weight of the cosmetic composition, although concentrations of up to about 20.0% can be used.

The overarching theory of this novel cosmetic treatment composition is to rescue senescent and pre-senescent cells rather than to execute them. This strategy has certain advantages over purely senolytic approaches, that become apparent in the following examples. The necessary components to implement this strategy are a cell-membrane stabilizing agent, a metabolic enhancing agent, an osmolyte stabilizing agent, collagen enhancing substances, and any minerals or nutrients that are likely to be deficient. Astaxanthin was chosen as the cell-membrane stabilizing agent since it also functions as a powerful antioxidant. Alpha-ketoglutarate was chosen as the metabolic enhancing agent since it has a wide range of beneficial effects. Glycine and the amino acid derivative trimethylglycine were chosen as the osmolyte stabilizing agents. Proline and Ornithine were chosen as the collagen enhancing substances. Bentonite clay was chosen to address mineral deficiencies. Vitamin A palmitate as well as niacinamide were chosen to address the most common nutritive deficiencies. When addressing deficiencies, it typically is not necessary to exceed 1.0% of the total weight of the cosmetic composition.

### Example 1

An ex-vivo skin study was conducted by Genemarkers LLC with DNA samples forwarded to CD Genomics for a methylation test using the Infinium MethylationEPIC v2.0 BeadChip. Skin samples were donated by a 65-year-old female subject from a cosmetic procedure. The samples were maintained ex-vivo for five days with two treatments of cream per day, one in the morning and one in the late afternoon. The untreated control samples were washed with PBS twice per day on the same schedule.

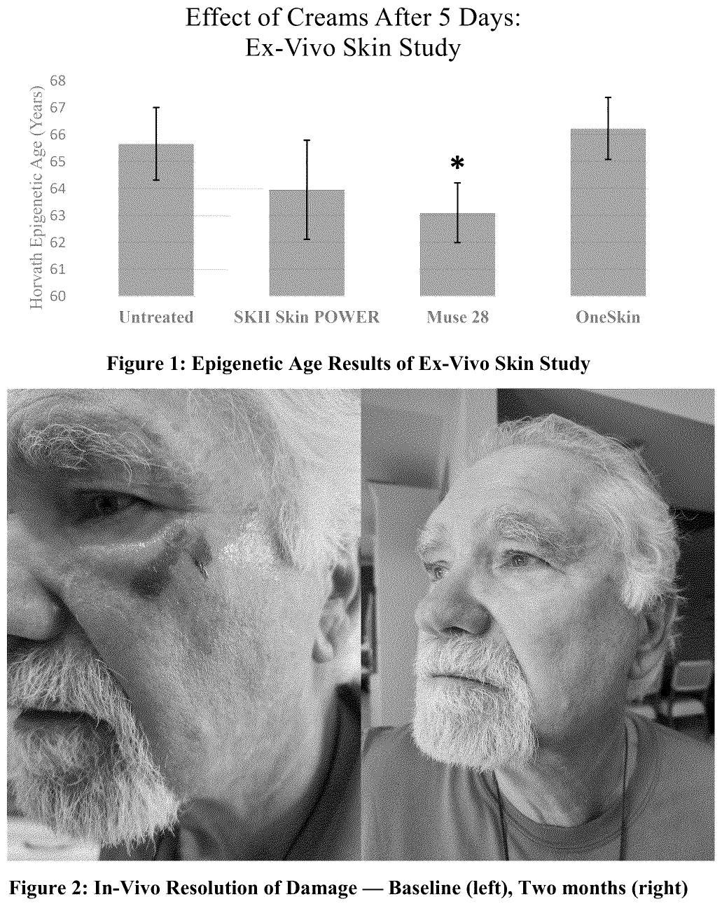

As shown in Table 1 and FIG. 1, the novel cosmetic preparation, “Muse 28”, reduced the average epigenetic age of the ex-vivo skin samples by 2.56 years. A Shapiro-Wilk test was conducted, and the underlying data may not have been normally distributed with p=0.0478. A skilled statistician will advise that it is better not to assume a normal distribution with a small sample size. Therefore, the non-parametric Wilcoxon Rank-sum test was used for statistical significance. Arguments were made for a Wilcoxon Signed-rank test, since the skin samples came from the same subject. However, the non-paired test was deemed appropriate since the DNA was derived from separate samples that were exposed to independent treatments. Only the novel cosmetic preparation met the threshold for statistical significance (*) with a p=0.0495.

TABLE 1

| | Average Horvath | Standard Deviation |
|---|---|---|
| Treatment | Epigenetic Age (Years) | (Years) |
| Untreated | 65.66 | ±1.35 |
| SKII Skin POWER | 63.95 | ±1.84 |
| Muse 28 | 63.10* | ±1.11 |
| OneSkin | 66.23 | ±1.15 |

These results were somewhat unexpected since the OneSkin product was intended to serve as a positive control. Zonari et al. reported a 2.5% reduction in epigenetic age using a senolytic compound described in US20210038729A1. However, this reduction was achieved in a 79-year-old subject. Despite having mentioned younger subjects in several public communications, no DNA methylation age data was presented for them. It is possible that the senolytic strategy alone is not sufficient to reduce age in subjects 65 or younger. Prior use of exfoliants, which were neither prevented nor encouraged, may also explain the ineffectiveness of the OneSkin product.

### Example 2

A neighbor's husband had minor complications following a laser procedure to remove precancerous cells on the face. He tried for several weeks to resolve the skin damage, but with a commercially available ceramide moisturizing cream recommended by his dermatologist, there was no visible improvement as shown in FIG. 2 (left). Significant repair was accomplished after one week of application of the novel cosmetic formulation, once per day. Two months later, as shown in FIG. 2 (right) there were no visible signs of scarring and the non-healing burn damage inflicted by the laser had been completely reversed.

### Example 3

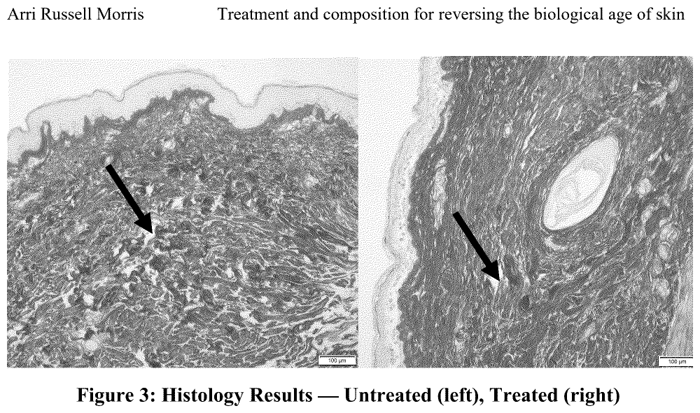

An ex-vivo skin study was conducted by Perfectus Biomed LLC. Skin samples were donated by a 49-year-old female subject from a cosmetic procedure. The samples were maintained ex-vivo for seven days with one treatment per day. The untreated control samples were washed with PBS. FIG. 3 (left) shows an untreated sample at 63× magnification from day one with a Picrosirius red stain to highlight collagen content. FIG. 3 (right) shows a sample at 63× magnification treated for seven days with the novel cosmetic composition, also stained with Picrosirius red. The tissue clumping indicated with the arrow on the left is not present in the treated sample. In the report, it was noted that the skin treated by the novel cosmetic composition demonstrated a “more robust colorization of collagen fibers and epidermis via picrosirius staining by qualitative visual assessment”, although no quantitative assessment was performed.

What is claimed is:

1. A method of treating skin comprising topical application to aged or damaged skin using a cosmetic composition comprising a chemically compatible combination of one or more restorative agents, in amounts effective to reverse the biological age of the skin's cells an average of 2.5 years in 5 days.

2. The method according to claim 1 wherein said combination includes both astaxanthin and alpha-ketoglutarate.

3. The method according to claim 1 wherein said treatment is effective to even skin tone and removing signs of sun or other burn damage.

4. The method according to claim 1 wherein said cosmetic composition comprises a chemically compatible combination of the following, in proportions based on the total weight of said cosmetic composition:
a) from about 0.01% to about 5.0% of astaxanthin;
b) from about 0.01% to about 10.0% of alpha-ketoglutarate;
c) from about 0.01% to about 5.0% of minerals and nutrients; and
d) from about 0.01% to about 15.0% of amino acids or amino acid derivatives.

5. The method of claim 1 wherein said chemically compatible combination comprises astaxanthin, alpha-ketoglutarate, amino acids, amino acid derivatives, minerals, and nutrients.

6. The method of claim 1 wherein said cosmetic composition has a pH in the range of from about 3.5 to about 5.5.

7. (canceled)

8. (canceled)

9. (canceled)

10. (canceled)

11. (canceled)

12. (canceled)

13. (canceled)

14. (canceled)

15. (canceled)

16. (canceled)
